(19) United States
(12) Patent Application Publication
(10) Pub. No.: US 2021/0038211 A1
(43) Pub. Date: Feb. 11, 2021

(54) SMART RETRACTOR BLADE

(71) Applicant: Zimmer Biomet Spine, Inc., Westminster, CO (US)

(72) Inventors: Edouard Jouan, Lavau (FR); David Rigotto, Saint Selve (FR); Florian Fontaine, Saint-Pouange (FR)

(21) Appl. No.: 16/983,595

(22) Filed: Aug. 3, 2020

Related U.S. Application Data
(60) Provisional application No. 62/882,676, filed on Aug. 5, 2019.

(51) Int. Cl. A61B 17/02; A61B 90/50; A61B 90/35; A61B 34/00

(52) U.S. Cl. CPC A61B 17/02; A61B 90/50; A61B 2090/064; A61B 34/25; A61B 90/35

(57) ABSTRACT

An instrument can be receivable in a coupler of a surgical arm. The instrument can include a stem, coupler, and a retractor blade. The stem can include a proximal portion couplable to a coupler, and can include an opposite distal portion. The retractor blade can be couplable to the coupler via a mating feature.

### CLAIM OF PRIORITY

This application claims the benefit of U.S. Provisional Patent Application Ser. No. 62/882,676, filed on Aug. 5, 2019, the benefit of priority of which is claimed hereby, and which is incorporated by reference herein in its entirety.

### BACKGROUND

The present invention relates generally to supported surgical instruments. Some surgical procedures include use of a variety of instruments. In some of these procedures, it is required that instruments, such as a retractor, be maintained in a single position for an extended period of time, such as an hour or more. During this time, other instruments can be used to perform other aspects of the surgery. Because it can be difficult or undesirable to manually hold a position of an instrument for such lengths of time, mechanical and/or electromechanical arms can be used to hold the position of the instrument. Some arms can be adjustable such that a position of the arm can be adjusted before or during the procedure.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an isometric view of a repositionable, lockable surgical arm, in accordance with at least one example of this disclosure.

FIG. 2 illustrates an exploded isometric view of a retractor, in accordance with at least one example of this disclosure.

FIG. 3A illustrates a side view of a retractor blade, in accordance with at least one example of this disclosure.

FIG. 3B illustrates a front view of a retractor blade, in accordance with at least one example of this disclosure.

FIG. 3C illustrates a rear isometric view of a retractor blade, in accordance with at least one example of this disclosure.

FIG. 3D illustrates a rear isometric view of a retractor blade, in accordance with at least one example of this disclosure.

FIG. 4A illustrates a front isometric view of a retractor blade, in accordance with at least one example of this disclosure.

FIG. 4B illustrates a side view of a retractor blade, in accordance with at least one example of this disclosure.

FIG. 5A illustrates a side view of a surgical arm system in a first condition, in accordance with at least one example of this disclosure.

FIG. 5B illustrates a side view of a surgical arm system in a second condition, in accordance with at least one example of this disclosure.

FIG. 5C illustrates a side view of a surgical arm system in a third condition, in accordance with at least one example of this disclosure.

FIG. 6 illustrates a graph, in accordance with at least one example of this disclosure.

FIG. 7 illustrates a rear isometric view of a retractor blade, in accordance with at least one example of this disclosure.

FIG. 8 illustrates a block diagram of a system, in accordance with at least one example of this disclosure.

### DETAILED DESCRIPTION

In some surgical procedures, it is required to hold instruments, such as a retractor, in a single position for an extended period of time, such as an hour or more. In these procedures, adjustable mechanical and/or electromechanical arms are often used to hold the position of the instrument while other aspects of the procedure are performed. However, forces required to retract the soft tissues (especially for those forces applied for relatively long durations) can cause patient discomfort following a procedure requiring such retraction.

This disclosure provides a solution to minimize patient discomfort by incorporating a load cell into a retractor that is usable with an adjustable arm, which allows for monitoring of applied forces and duration. The load cell can communicate with a controller (and a user interface in some examples) to communicate a force applied by the retractor, A duration that the force is applied can also be monitored and logged. Alarms can be produced when a threshold force is exceeded by the retractor and/or a threshold duration of force application is exceeded. In some examples, the controller can communicate with an electromechanical arm to automatically reduce the force applied by the retractor based on a threshold force and/or threshold duration. The force applied by the arm can also be limited by the controller.

The above discussion is intended to provide an overview of subject matter of the present patent application. It is not intended to provide an exclusive or exhaustive explanation of the invention. The description below is included to provide further information about the present patent application.

As used herein, the terms “proximal” and “distal” should be given their generally understood anatomical interpretation. The term “proximal” refers to a direction generally toward the torso of a patient or base or handle of a tool or instrument, and “distal” refers to the opposite direction of proximal, i.e., away from the torso of a patient or toward the working end of the tool.

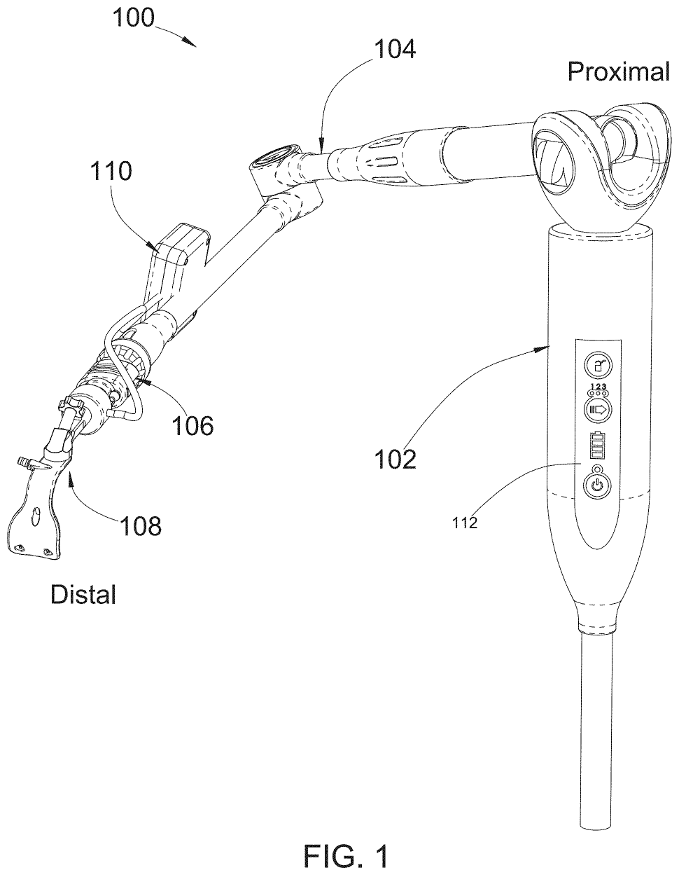

FIG. 1 illustrates a perspective view of a surgical arm system 100, in accordance with at least one example of this disclosure. The surgical arm 100 can include a control device 102, an arm 104, a coupler 106, an instrument 108, and a user interface 110. The control device 102 can include a primary user interface 112. Also shown in FIG. 1 are orientation indicators Proximal and Distal.

The control device 102, which can include power control circuit components for an electrically powered actuator, can be secured to a rail of a surgical table using, for example, a clamp. The user interface 112 of the control device can include buttons and/or switches for operating the surgical arm and can optionally include a screen that can display information about the status of the arm. In some examples, the arm 104 can include one or more locking joints and an actuator.

The coupler 106 can be, for example, an end effector coupler, used to quickly and easily remove and secure tools and instruments, such as the instrument 108, to the surgical arm 104, A button 116 can be integrated on the coupler 106, where operation of the button 116 can lock and unlock the surgical arm 104. The instrument 108 can be a surgical retractor configured to engage and retract soft tissue, such as skin and muscle. In one example, the instrument can be configured to retract an oesophagus, such as during a spinal operation using an anterior cervical approach. The instrument 108 can be connected to the user interface 110 either directly or indirectly, such as through the control unit 102. The user interface 110 can be configured to display information regarding the instrument 108 to a user and can be configured to receive inputs from a user in some examples. The user interface 110 is discussed in further detail below.

In operation of some examples, the primary user interface 112 can be operable by a user to initiate power locking and unlocking of the arm 104. When a lock/unlock button is not depressed, the arm 104 can be in a locked state where joints are locked such that proximal and distal links of the arm 104 cannot move relative to each other or to the table, coupler 106, or patient. When the lock/unlock button is pressed, an actuator can unlock joints of the arm 104 such that the coupler 106 and the instrument 108 can be positioned, as desired, and as guided by the arm 104. (In some examples, an actuator can be omitted and the arm joints can be individually lockable.) Re-locking the arm 104 will again lock the position of the arm 104. Such a process can be repeated, as desired. In some examples, the arm can include one or more actuators to move the arm 104 without force applied to the arm 104 by the user. For example, the joints of the arm 104 can include actuators in communication with the control unit 102.

During positioning of the arm 104, the instrument 108 can be used, for example, as a retractor. When the arm 104 is unlocked, the instrument 108 can be moved to retract tissue and the arm 104 can be locked to hold the instrument 108 in a position such that the tissue remains in a retracted position as desired. As discussed in further detail below, a load cell can be attached to the instrument 108 and can transmit signals to the user interface 110 and/or the primary user interface 112. The user interface 110 can display information about the instrument 108 and its operation, such as force applied to the instrument 108 and duration of the application of the force. In some examples, the arm 104 can automatically unlock in response to a force signal above a threshold and/or a force held above the threshold for a duration. In other examples, the control unit 102 and/or 112 and/or interface 110 can produce an alert when the threshold force is reached and/or a force is applied for a specified duration.

In some examples, the arm 104 can be prevented from locking in response to a force signal above a threshold and/or a force held above the threshold for a duration. In other examples, the control unit 102 and/or 112 and/or 110 can produce an alert when locking is attempted and the threshold force is reached. Further details regarding the instrument 108 and its use are discussed below.

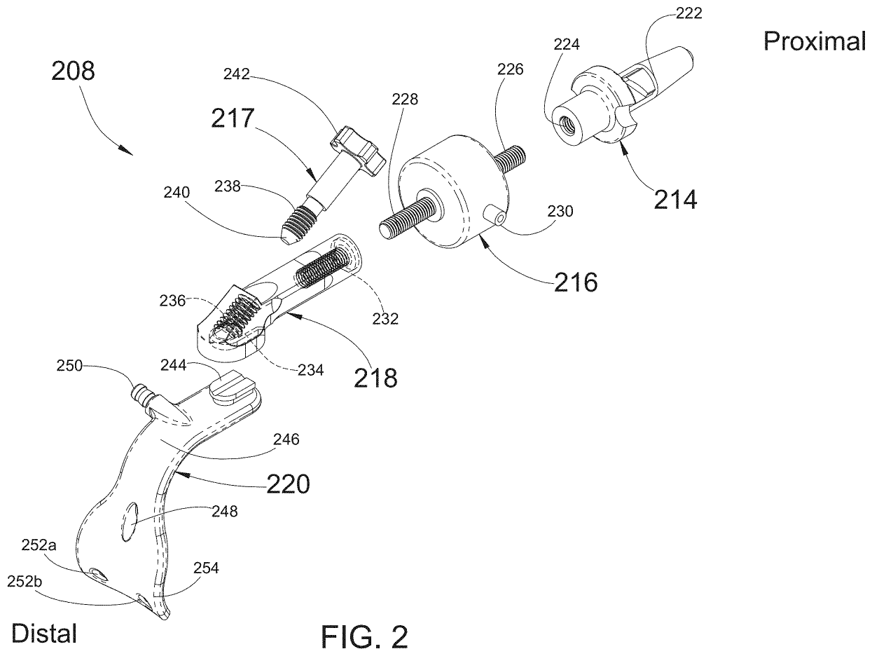

FIG. 2 illustrates an exploded isometric view of a retractor 208, in accordance with at least one example of this disclosure. The retractor 208 can include a stem 214, a load cell 216, a set screw 217, a coupler 218, and a retractor blade 220. Also show in FIG. 2 are orientation indicators Proximal and Distal.

Each of the components of the retractor 208 can be comprised of materials such as metals, plastics, foams, elastomers, ceramics, composites, or combinations thereof. In some examples, the retractor blade 220 can be made of radiolucent materials. In some examples, the components, such as the retractor blade 220, can be comprised of biocompatible materials, such as one or more of stainless steels, cobalt-chromium, titanium variations, polyether ether ketone (PEEK), other plastics, or the like.

The stem 214 can include a proximal portion 222 couplable to an end effector coupler (such as the coupler 106 of FIG. 1), and an opposite distal portion 224. The distal portion 224 can include a mating feature, such as threaded bore; the mating feature can be other types of mating features, such as one or more pins, clamps, clips, or the like. The mating feature of the distal portion 224 can be configured to receive a proximal threaded rod 226 of the load cell 216.

The load cell 216 can include the proximal threaded rod 226 and a distal threaded rod 228 for releasable threaded coupling to the distal portion 224 of the stem 214 and the coupler 218, respectively. The load cell 216 can be a sensor configured to produce a signal as a function of a measured force, such as a hydraulic load cell, pneumatic load cell, strain load cell, or the like. The load cell 216 can include a connector 230 configured to receive a tube, wire, or the like for connecting the load cell 216 to a user interface and/or a controller.

The coupler 218 can include a proximal bore 232 configured to receive the distal threaded rod 228 of the load cell 216 to couple the coupler 218 to the load cell 216. The proximal bore 232 can be other types of mating features, such as one or more pins, clamps, clips, or the like. The coupler 218 can also include a coupler mating feature 234, which can be a slot or channel, in some examples. The coupler 218 can further include a set screw bore 236 sized to receive a portion of the set screw 217 therein. The set screw bore 236 can intersect the mating feature 234, such as a slot 234.

The set screw 217 can include a threaded portion 238 securable within the set screw bore 236 of the coupler. The set screw 217 can also include a tip 240 engageable with a boss 244 of the retractor blade 222. The set screw 217 can further include a handle 242 operable to rotate the set screw 217 about its axis and operable to position (such as by screwing) the set screw 217 into and out of the set screw bore 236 and into the slot 234, and therefore operable to engage and disengage the boss 244 of the retractor blade 222 to limit translation of the boss 244 within the slot 234 when the boss 244 is positioned in the slot 234.

The retractor blade 220 can include a body 246 configured to support the boss 244, a light 248, cannula inlets 252a and 252b, a distal portion 254, and the canula 250. The boss 244 can extend from an outer surface of the body 246 and can be sized and shaped to engage the slot 234 of the coupler 218. The light 248 can be a light emitting diode (LED), or other type of light, positioned within the body 246 to emit light in a distal direction during operation of the retractor blade 220. The light 248 can include an opening in the body 246 that supports the LED and supporting wiring. The suction inlets 252 can be openings in the body 246 at the distal portion 254 of the body 246. The suction inlets 252 can be connected to channels within the body 246 that connect to a cannula outlet 250. The cannula outlet 250 can be configured to be connected to a suction source (such as a pump) via tubing or piping within a surgical suite or room.

In operation of assembly, the proximal threaded rod 226 of the load cell 216 can be secured to the distal portion 224 of the coupler 218 and the distal threaded rod 22.8 can be secured to the proximal bore 232 of the coupler 218. The boss 244 can be inserted into the slot 234 of the coupler 218 and positioned therein (such as by translating the boss 244 within the slot 234). When a desirable position of the retractor blade 220 with respect to the coupler, the handle 242 can be operated. to insert the tip 240 and threaded portion 238 of the set screw within the set screw bore 236. The handle 242 can be further operated to secure the threaded portion 238 to the set screw bore 236 (such as through a threaded engagement) until the tip 240 engages the boss 244, where the engagement between the tip 240 and the boss 244 can limit movement of the boss 244 (and therefore the retractor blade 220) with respect to the coupler 218, Either prior to assembly of the retractor 208 or after, the proximal portion 222 of the coupler 214 can be secured to a coupler of a surgical arm (such as an end effector coupler for example, the coupler 106 of FIG. 1).

Once the retractor 208 is assembled and connected to a surgical arm, the distal portion 254 of the retractor 208 can be used to engage tissue of a patient for retraction of the tissue, where the retractor 208 and the arm can hold the tissue in a retracted position, as desired. When the distal portion 254 engages the tissue, a force can be applied to the distal portion 254 by the tissue, as discussed in further detail below. In one example, the retractor blade 220, together with the stem 214, the load cell 216, and the coupler 218 can be movable with an end effector coupler (such as the coupler 106 of FIG. 1), where the load cell 216 can be configured to produce a load signal based on a force applied to the retractor blade 220 by the tissue. The signal can be transmittable from the load cell 216 to one or more controllers.

After engagement with the tissue, the retractor 208 can be moved or repositioned as desired during a procedure. The signal transmittable from the load cell 216 can change as the force applied to the retractor blade 220 by the tissue changes. The signal can be used to produce alerts or help control the surgical arm to which the retractor 208 is attached. When desired, the retractor blade 220 can be disengaged from the tissue.

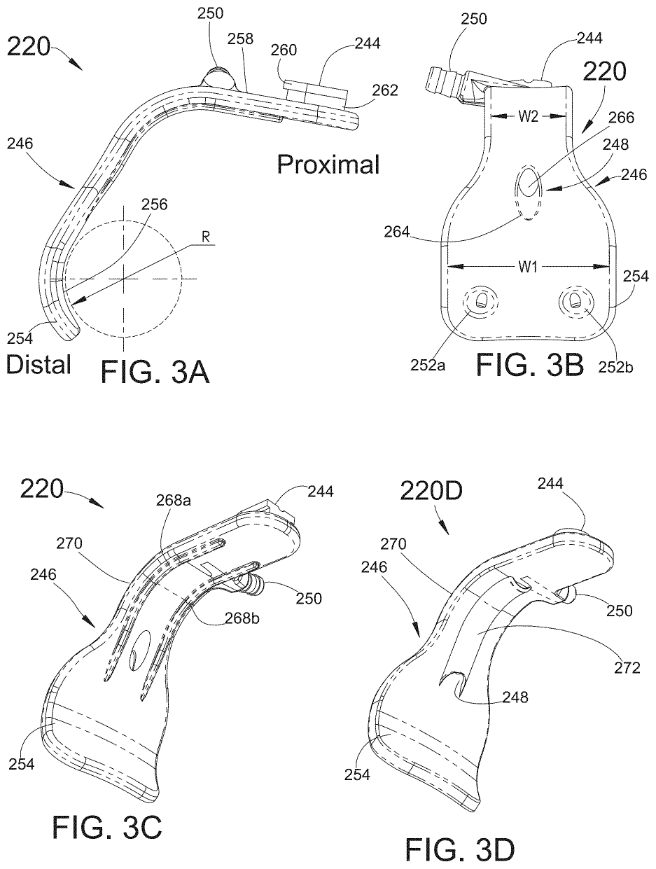

FIG. 3A illustrates a side view of the retractor blade 220, in accordance with at least one example of this disclosure. FIG. 3B illustrates a front view of the retractor blade 220, in accordance with at least one example of this disclosure. FIG. 3C illustrates a rear isometric view of the retractor blade 220, in accordance with at least one example of this disclosure. FIGS. 3A-3C are discussed below concurrently.

The retractor blade 220 can be consistent with the retractor blade 220 discussed with respect to FIGS. 1 and 2 above; additional details of the retractor blade 220 are discussed below with respect to FIGS. 3A-3C. For example, FIG. 3A shows that the distal portion 254 of the retractor blade can have a curved portion 256 having a radius of curvature R, where the radius of curvature R is sized for retraction of a specific type of tissue of a patient. For example, radius of curvature R can be sized to retract an oesophagus (esophagus), that is the radius of curvature R can be adapted to the tracheo-esophageal complex to help reduce pain during and after a procedure. In some examples, the radius of curvature R can be between 10 and 30 millimeters. In some examples, the radius of curvature R can be about 20 millimeters. In other examples, the radius of curvature R can be between 1 and 100 millimeters. In other examples, the retractor blade can be configured for retraction of tissues in other procedures, such as lumbar retraction (anterior or lateral approach) ; nerve root retraction, vessel retraction, and/or brain tissue retraction.

FIG. 3A also shows that the boss 244 can extend from a top surface 258 of the retractor blade 220 where a lip 260 can extend radially outward from a base 262 of the boss 244. The lip 260 can be sized to be larger than the slot 234 such that the boss 244 can be retained within the slot 234 of the connector 218 as the boss 244 moves between a locked (proximal) position and an unlocked (distal) position with respect to the coupler 218.

FIG. 3B shows that the light 248 can include a bore 264 extending into the body 246 where an LEI) 266 can be located within the bore 264. In some examples, the light 248 can be positioned on the body 246 between the cannula inlets 252a and 252b (or first and second channels 242a and 252b, respectively), In some examples, the light 248 can be positioned on the distal blade portion 254 between the cannula inlets 252a and 252b.

FIG. 3B also shows that the distal portion 254 can have a width W1 substantially wider than a width W2 of an upper portion of the body 246. The larger width W1 can help reduce a pressure applied by the retractor blade 220 on tissue of the patient during retraction. FIG. 3C shows that the body 246 can include reinforcements 268a and 268b which can help reinforce a curved portion 270 of the body 246 to help limit plastic deformation of the body 246 during retraction.

FIG. 3D illustrates a rear isometric view of a retractor blade 220D, in accordance with at least one example of this disclosure. The retractor blade 220D can be similar to the retractor blade 220 discussed above with respect to FIGS. 2-3C except that the retractor blade 220D can include a single reinforcement 272 to help reinforce the under-side of the curved portion 270 of the body 246. In some examples, the single reinforcement 272 can also act as a wire chase or channel for supporting power and/or control wires connected to the light 248.

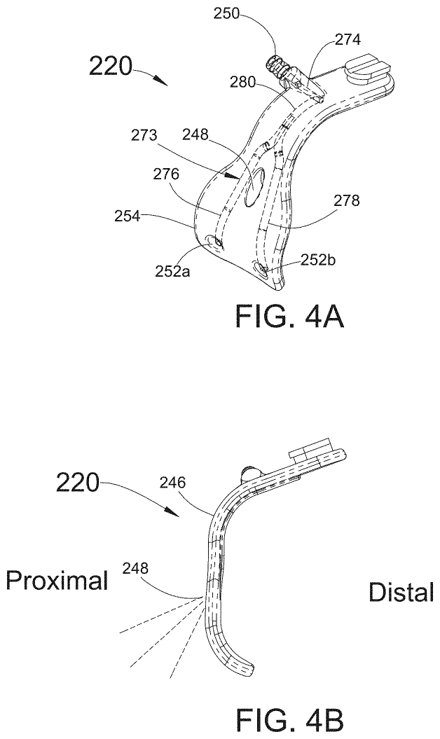

FIG. 4A illustrates a front isometric view of the retractor blade 220, in accordance with at least one example of this disclosure. FIG. 4B illustrates a side view of the retractor blade 220, in accordance with at least one example of this disclosure. FIGS. 4A and 4B show orientation indicators Proximal and Distal and are discussed below concurrently.

The retractor blade 220 can be consistent with the retractor blade 220 discussed above with respect to FIGS. 1-3C; FIGS. 4A and 4B show additional details of the retractor blade 220. For example, FIG. 4A shows that body 246 of the retractor can include multiple channels. More specifically, FIG. 4A shows the cannula 250 connected to the retractor blade, shows the first cannula inlet 252a extending through the distal blade portion 254 of the body 246, and shows the second cannula inlet 252b extending through the distal blade portion 254.

FIG. 4A also shows a cannula conduit 273 extending through the retractor blade. The cannula conduit can connect to the first cannula inlet 252a, the second cannula inlet 252b, and the cannula 250. The cannula conduit 273 can include a first channel 274 connected to the cannula 250 and extending into the distal portion 254 of the retractor blade 220. A second channel 276 can be connected to the first channel 274 and the first cannula inlet 252a. A third channel 278 can be connected to the first channel 274, the second channel 276, and the second cannula inlet 252b. In some examples, a fourth channel 280 can connect the first channel 274, the second channel 276, and the third channel 278.

In operation, the cannula outlet 250 can be connected to a suction line (such as a tube or pipe) to connect the cannula conduit 273 to a pump that applies suction pressure to the cannula conduit 273. The cannula conduit 273 can apply the negative pressure to the first channel 274, the second channel 276, the third channel, the fourth channel 280, the first cannula inlet 252a, and the second cannula inlet 252b. The cannula inlets 252 can thereby apply suction pressure to a cavity of the patient to remove fluids and/or smoke, for example, from the cavity.

The first channel 252a and the second channel 252b can extend through the body 246 of the retractor blade 220 around the light 248 to avoid interference with the light 248 and the wiring to the light 248. Though two cannula inlets are shown are shown and discussed, the retractor blade 220 can include 1, 3, 4, 5, 6, 7, 8, 9, 10, or the like cannula inlets. FIG. 4B shows that the light 248 can project proximally from the body 246 of the retractor blade 220 to help improve viewing of a surgical site of the patient.

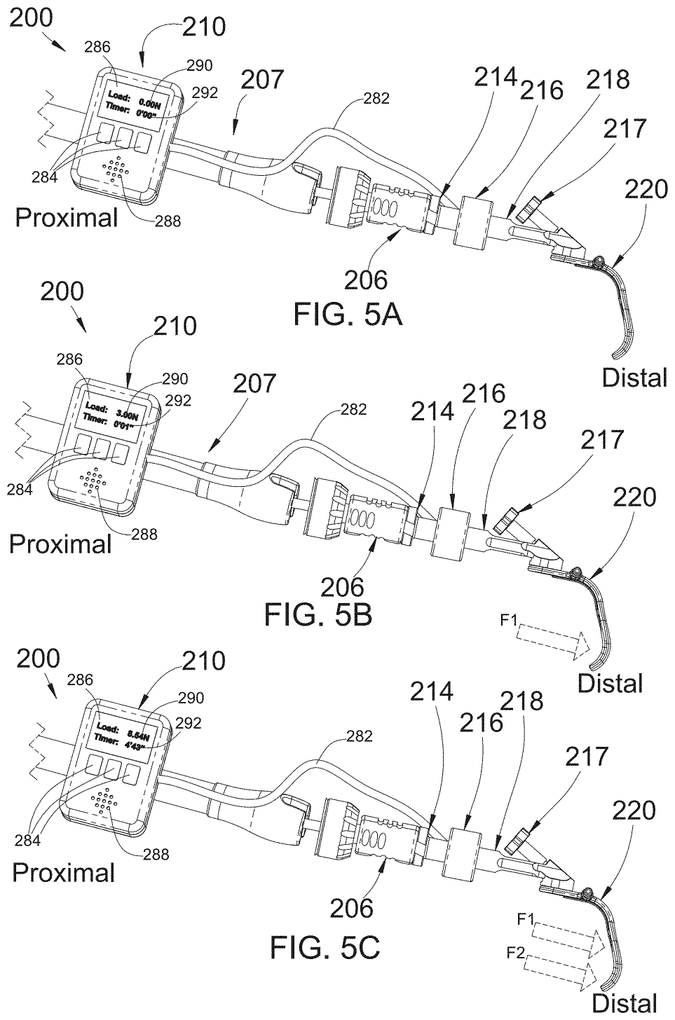

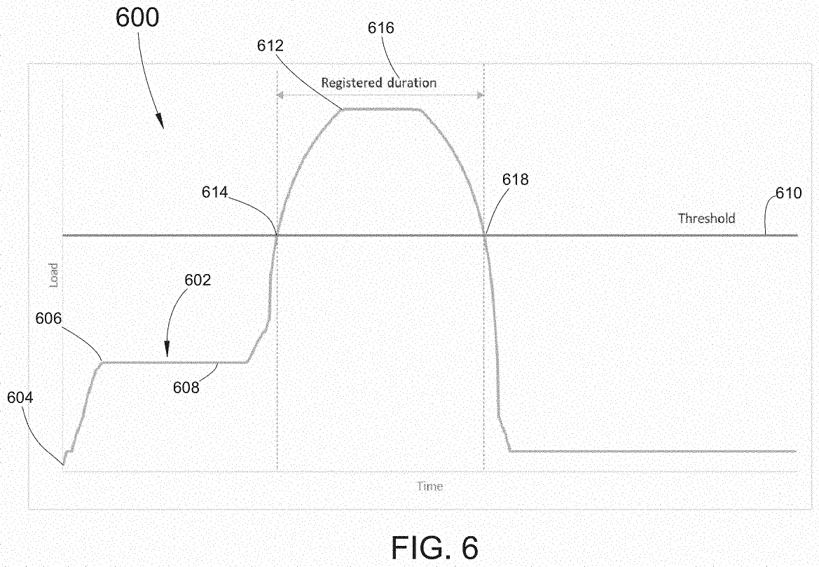

FIG. 5A illustrates a side view of a surgical arm system 200 in a first condition, in accordance with at least one example of this disclosure. FIG. 5B illustrates a side view of the surgical arm system 200 in a second condition, in accordance with at least one example of this disclosure. FIG. 5C illustrates a side view of the surgical arm system 200 in a third condition, in accordance with at least one example of this disclosure. FIG. 6 illustrates a graph 600, in accordance with at least one example of this disclosure. FIGS. 5A-5C and 6 are discussed below concurrently. FIGS. 5A-5C show forces F1 and F2 and orientation indicators Proximal and Distal.

The components of the surgical arm system 200 can be consistent with those discussed above; FIGS. 5A-5C show additional details of the surgical arm system 200 and show the surgical arm system 200 in various conditions. For example, FIGS. 5A-5C shows that the load cell 216 can be connected to the controller 210 via a conduit 282, which can be one or more wires. FIGS. 5A-5C also show that the controller 210 can include controls 284, a display 286, and a speaker 288. The controls 284 can be touch-sensitive controls, buttons, or the like configured to receive user inputs. The display 286 can be a screen such as a liquid crystal display (LCD), plasma screen, or the like configured to display output from the controller 210.

In operation of some examples, once the surgical arm system 200 is assembled (as discussed above with respect to FIG. 2), the system 200 can be used during an operation or procedure. When the retractor blade 220 is not engaging any tissue, the load cell 216 will not have a force applied thereto and will therefore produce a signal indicating that no force is applied to the load cell 216. The control 210 can interpret that signal and modify the outputs shown on the display 286. For example, a load 290 can indicate a force of 0 N (0 Newtons) and a time 292 can indicate that no time duration has passed where a load is applied. This point is shown as point 604 on the graph 600 near the origin, where the line 602 indicates a force applied for a given time. Such a graph can be displayed on the user interface 286 or on any other user interface that can be in communication with the controller 210.

As shown in FIG. 5B, a force F1 can be applied to the retractor blade 220, which can be transferred to the load cell 216. The load cell 216, in response to the force F1, can produce a signal transmitted through the conduit 282 to the controller 210. The controller 210 can modify the load 290 to display, for example, 3.00 Newtons, and can modify the timer 292 to display 0′01″ (0 minutes and 1 second). This point can be represented on the graph 600 by point 606. As the force F1 is continually applied to the retractor blade, the force can remain constant, as shown as a line 608 on the graph 600, where the line 608 is below a threshold 610. The threshold 610 can be a threshold force where damage may occur to a tissue if applied for a specified duration of time. In some examples, multiple thresholds can be accounted for.

As shown in FIG. 5C, an additional force F2 can be applied to the retractor blade 220 (or, a larger force F1 can be applied to the retractor blade 220, in other examples). The second force F2 can combine with the first force F1 to create a force that registers, for example, 8.54 Newtons, as shown on the load 290 of the display. The timer 292 can show that the load has been applied for a time of 4′43″ (or 4 minutes and 43 seconds). Such a force application can be indicated by point 612 of the graph 600 where the combined forces F1 and F2 are above the threshold 610, which can be, for example, 7 Newtons. Other threshold values can be used.

In some examples, the controller 210 can produce an alert when the force (indicated by line 602) rises above the threshold, such as at point 614 of the graph 600. The alert can be an audible alert using the speaker 288, a visual alert using the display 286, or other alerts produced through one or more additional displays, such as an operating room monitor, wearable device, or the like.

In some examples, the controller can produce an alert when the force is applied at or above a threshold level 610 for a specified duration or longer, where the threshold force and/or the threshold time are indicative of a likelihood of creating post-operative pain. For example, the force indicated by line 602 is above the threshold given at point 614 and does not drop below the threshold 610 until point 618. If the duration of the force is greater than a predetermined duration, an alert can be produced indicating as much.

In some examples, when either of these conditions (force above threshold or force and duration above threshold) exist, the controller 210 and/or another controller (such as the controller 102 of FIG. 1) can move the arm to relive the force applied by the retractor blade 220 to the tissue under retraction. For example, one of the controllers can unlock the joints of the arm, which can relive the force applied by the retractor blade 220.

In one example, the controller 210 can be configured to transmit a control signal to the surgical arm 200 to reduce the force applied to the retractor blade 220 when a second threshold force is exceeded for a second threshold time. For example, a threshold of 10 Newtons for 10 minutes.

In another example, the controller 210 can configured to control a color or intensity, based on the load signal, of a light emitted by the light 248 connected to the retractor blade 220 to indicate whether the threshold force is reached. For example, the light can be white when the threshold force is not reached, can be yellow when the threshold force is reached, and can be red when the threshold force is held beyond the threshold duration.

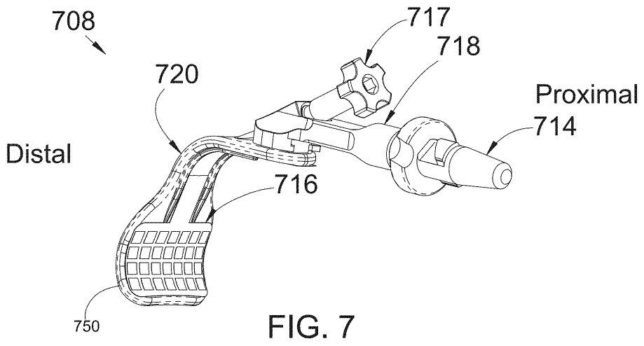

FIG. 7 illustrates a rear isometric view of a retractor 708, in accordance with at least one example of this disclosure. The retractor 708 can be similar to the retractors 108 and 208 discussed above, except that the retractor 708 can include a mapping patch 716 secured to the retractor blade 720. The retractors 108 and 208 discussed above can be modified to include such a mapping patch.

The mapping patch 716 can be positioned on proximal side of a distal portion 754 of the retractor blade 720, such as at a portion of the blade configured to contact tissue. The mapping patch 716 can include an array of force and/or pressor sensors configured produce one or more signals based on forces or pressures applied to the various sensors of the mapping patch 716 by engagement with, for example, patient tissue. In some examples, the mapping patch 716 can include a plurality of electrodes arranged in an array to measure a pressure gradient or distribution.

The signal(s) produced can be transmitted to one or more controllers and used to determine a location and intensity of one or more pressures applied to the mapping patch 716. The values can be used to create visual displays such as a. display indicative of loading on the retractor blade 720. The output can also be used to produce an alert that indicates when the retractor blade 720 should be repositioned, such as based on one or more pressures and durations of application of such pressures.

In some examples, the mapping patch 716 can be disposable and in other examples, the mapping patch 716 can be reusable. In some examples, the mapping patch 716 can be calibrated to a specific task, such as retraction of an oesophagus. In some examples, the mapping patch 716 can be customized to a desired resolution and size based on engagement of the patch 716 with a specification body part, such as retraction of an oesophagus.

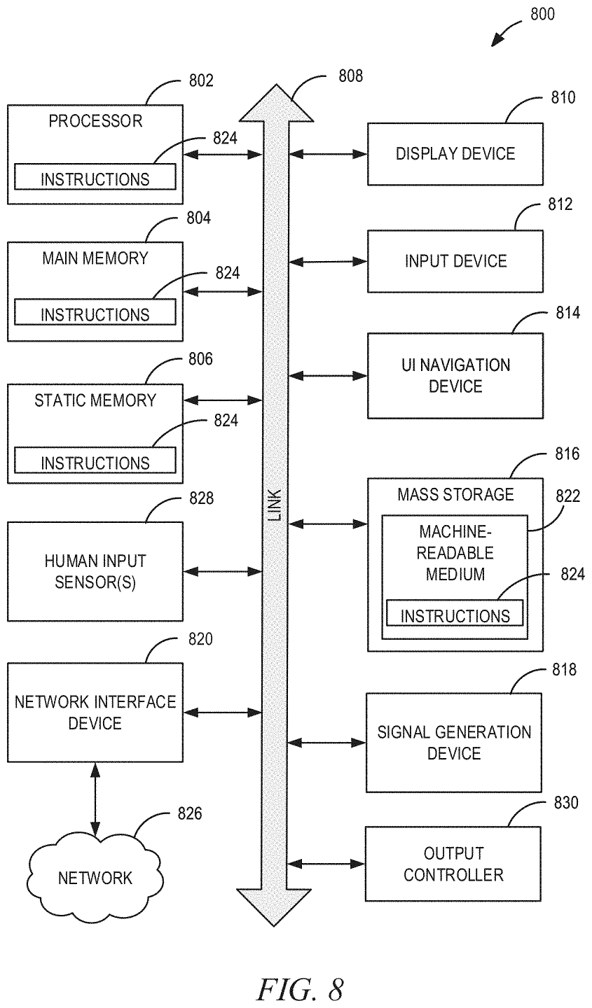

FIG. 8 illustrates a block diagram of an example system or machine 800 upon which any one or more of the techniques (e.g., methodologies) discussed herein may perform. The machine 800 can be one or more of the controllers discussed above (120, 110, 210, etc.) Examples, as described herein, may include, or may operate by, logic or a number of components, or mechanisms in the machine 800. Circuitry (e.g., processing circuitry) is a collection of circuits implemented in tangible entities of the machine 800 that include hardware (e.g., simple circuits, gates, logic, etc.), Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time, Additional examples of these components with respect to the machine 800 follow.

In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 800 may include a hardware processor 802 (e.g., a central processing unit (CPU), a graphics processing unit (GPU), a hardware processor core, or any combination thereof), a main memory 804, a static memory (e.g., memory or storage for firmware, microcode, a basic-input-output (BIOS), unified extensible firmware interface (UEFI), etc.) 806, and mass storage 808 (e.g., hard drive, tape drive, flash storage, or other block devices) some or all of which may communicate with each other via an interlink (e.g., bus) 830. The machine 800 may further include a display unit 810 (which can be similar to the display 286), an alphanumeric input device 812. (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 808, a signal generation device 818 (e.g., a speaker, such as the speaker 288), a network interface device 820, and one or more sensors 816, such as the load sensor 216 or mapping sensor 716. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 802, the main memory 804, the static memory 806, or the mass storage 808 may be, or include, a machine readable medium 822 on which is stored one or more sets of data structures or instructions 824 (e.g., software) embodying or utilized by any one or more of the techniques or functions described herein. The instructions 824 may also reside, completely or at least partially, within any of registers of the processor 802, the main memory 804, the static memory 806, or the mass storage 808 during execution thereof by the machine 800. In an example, one or any combination of the hardware processor 802, the main memory 804, the static memory 806, or the mass storage 808 may constitute the machine readable media 822. While the machine readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may be further transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software. A transmission medium is a machine readable medium.

### NOTES AND EXAMPLES

The following, non-limiting examples, detail certain aspects of the present subject matter to solve the challenges and provide the benefits discussed herein, among others.

Example 1 is an instrument receivable in an end effector coupler of a surgical arm, the instrument comprising: a stem including a proximal portion couplable to an end effector coupler, and an opposite distal portion; a load cell couplable to the distal portion of the stem; a coupler couplable to the load cell, the coupler including a mating feature; and a retractor blade couplable to the coupler via the mating feature, the retractor blade, together with the stem, the load cell, and the coupler, movable with the end effector coupler, the load cell configured to produce a load signal based on a force applied to the retractor blade.

In Example 2, the subject matter of Example 1 includes, millimeters.

In Example 3, the subject matter of Examples 1-2 includes, a cannula outlet connected to the retractor blade; a first cannula inlet extending through a distal blade portion of the retractor blade; a second cannula inlet extending through the distal blade portion; and a cannula conduit extending through the retractor blade, the cannula conduit comprising: a first channel connected to the cannula outlet and extending into the distal blade portion; a second channel connected to the first channel and the first cannula inlet; and a third channel connected to the first channel, the second channel, and the second cannula inlet.

In Example 4, the subject matter of Example 3 includes, a light located on the distal blade portion positioned between the first channel and the second channel.

In Example 5, the subject matter of Example 4 includes, wherein the first channel and the second channel extend through the retractor blade around the light.

In Example 6, the subject matter of Examples 1-5 includes, wherein: the mating feature of the coupler comprises a slot; and a proximal portion of the retractor blade comprises a raised boss insertable into the slot of the mating feature and translatable therein to move the retractor blade between a locked position and an unlocked position with respect to the coupler.

In Example 7, the subject matter of Example 6 includes, a set screw positionable through the coupler into the slot, the set screw adjustable to engage the raised boss to limit translation of the boss within the slot.

In Example 8, the subject matter of Example 7 includes, wherein: the coupler includes a threaded bore intersecting the slot; and the set screw includes a handle operable to thread the set screw in and out of a threaded bore of the coupler to engage and disengage the raised boss when the raised boss is positioned in the slot of the mating feature.

In Example 9, the subject matter of Examples 1-8 includes, the retractor blade further comprising: a proximal blade portion couplable to the coupler; a curved portion connecting the proximal blade portion and the distal blade portion, the distal blade portion curved to engage tissue of a patient for retraction; and a light located on the distal portion.

In Example 10, the subject matter of Examples 7-9 includes, a wiring channel connected to an under-side of the curved portion and connecting the light, the wiring channel configured to reinforce the curved portion of the blade.

Example 11 is an instrument receivable in an end effector coupler of a surgical arm, the instrument comprising: a stem including a proximal portion couplable to an end effector coupler, and an opposite distal portion; a load cell couplable to the distal portion of the stem and configured to produce a load signal based on a force applied to the load cell; a coupler couplable to the load cell, the coupler including a mating feature; a retractor blade couplable to the coupler via the mating feature, the retractor blade configured to transfer a force applied to the retractor blade to the load cell via the coupler; and a controller in communication with the load cell, the controller configured to modify the load cell signal.

In Example 12, the subject matter of Example 11 includes, a user interface in communication with the controller and configured to produce a display based on the load cell signal from the controller.

In Example 13, the subject matter of Examples 11-12 includes, wherein the controller is configured to produce an alert based on the load cell signal when a threshold force is exceeded for a threshold time.

In Example 14, the subject matter of Example 13 includes, wherein the threshold force and the threshold time are indicative of a likelihood of creating post-operative pain.

In Example 15, the subject matter of Examples 13-14 includes, wherein the controller is configured to transmit a control signal to a surgical arm connected to the end effector coupler to reduce the force applied to the retractor when the threshold force is exceeded for the threshold time.

In Example 16, the subject matter of Examples 13-15 includes, wherein the controller is configured to transmit a control signal to a surgical arm connected to the end effector coupler to reduce the force applied to the retractor when a second threshold force is exceeded for a second threshold time.

In Example 17, the subject matter of Examples 13-16 includes, wherein the controller is configured to control a color or intensity, based on the load signal, of a light emitted by a light connected to the retractor blade.

Example 18 is an instrument receivable in an end effector coupler of a surgical arm, the instrument comprising: a stem including a proximal portion couplable to an end effector coupler, and an opposite distal portion; a coupler securable to the load cell, the coupler including a mating feature; and a retractor blade couplable to the coupler via the mating feature, the retractor blade, together with the stem and the coupler, movable with the end effector coupler.

In Example 19, the subject matter of Example 18 includes, wherein: the mating feature of the coupler comprises a slot; and a proximal portion of the retractor blade comprises a raised boss insertable into the slot of the mating feature and translatable therein to move the retractor blade between a locked position and an unlocked position with respect to the coupler.

In Example 20, the subject matter of Examples 18-19 includes, a set screw positionable through the coupler into the slot, the set screw adjustable to engage the raised boss to limit translation of the boss within the slot.

In Example 21, the subject matter of Examples 18-20 includes, a load cell positioned on the retractor blade and configured to produce a load signal based on a force applied to the load cell and the retractor blade.

Example 22 is at least one machine-readable medium including instructions that, when executed by processing circuitry, cause the processing circuitry to perform operations to implement of any of Examples 1-21.

Example 23 is an apparatus comprising means to implement of any of Examples 1-21.

Example 24 is a system to implement of any of Examples 1-21.

Example 25 is a method to implement of any of Examples 1-21.

In Example 26, the apparatuses or method of any one or any combination of Examples 1-25 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof, either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” in this document, the term “of” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure, It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should he determined with reference to the appended claims, along with the full scope of equivalents to which such claims are entitled.

1. An instrument receivable in an end effector coupler of a surgical arm, the instrument comprising:
a stem including a proximal portion couplable to an end effector coupler, and an opposite distal portion;
a load cell couplable to the distal portion of the stem;
a coupler couplable to the load cell, the coupler including a mating feature; and
a retractor blade couplable to the coupler via the mating feature, the retractor blade, together with the stem, the load cell, and the coupler, movable with the end effector coupler, the load cell configured to produce a load signal based on a force applied to the retractor blade.

2. The instrument of claim 1, wherein the retractor blade includes a curved distal portion having a radius of curvature of about 20 millimeters.

3. The instrument of claim 1, further comprising:
a cannula outlet connected to the retractor blade;
a first cannula inlet extending through a distal blade portion of the retractor blade;
a second cannula inlet extending through the distal blade portion; and
a cannula conduit extending through the retractor blade, the cannula conduit comprising:
a first channel connected to the cannula outlet and extending into the distal blade portion;
a second channel connected to the first channel and the first cannula inlet; and
a third channel connected to the first channel, the second channel, and the second cannula inlet.

4. The instrument of claim 3, further comprising:
a light located on the distal blade portion positioned between the first channel and the second channel.

5. The instrument of claim 4, wherein the first channel and the second channel extend through the retractor blade around the light.

6. The instrument of claim 1, wherein:
the mating feature of the coupler comprises a slot; and
a proximal portion of the retractor blade comprises a raised boss insertable into the slot of the mating feature and translatable therein to move the retractor blade between a locked position and an unlocked position with respect to the coupler.

7. The instrument of claim 6, further comprising:
a set screw positionable through the coupler into the slot, the set screw adjustable to engage the raised boss to limit translation of the boss within the slot.

8. The instrument of claim 7, wherein:
the coupler includes a threaded bore intersecting the slot; and
the set screw includes a handle operable to thread the set screw in and out of a threaded bore of the coupler to engage and disengage the raised boss when the raised boss is positioned in the slot of the mating feature.

9. The instrument of claim 1, the retractor blade further comprising:
a proximal blade portion couplable to the coupler;
a curved portion connecting the proximal blade portion and the distal blade portion, the distal blade portion curved to engage tissue of a patient for retraction; and
a light located on the distal portion.

10. The instrument of claim 7, further comprising a wiring channel connected to an under-side of the curved portion and connecting the light, the wiring channel configured to reinforce the curved portion of the blade.

11. An instrument receivable in an end effector coupler of a surgical arm, the instrument comprising:
a stem including a proximal portion couplable to an end effector coupler, and an opposite distal portion;
a load cell couplable to the distal portion of the stem and configured to produce a load signal based on a force applied to the load cell;
a coupler couplable to the load cell, the coupler including a mating feature;
a retractor blade couplable to the coupler via the mating feature, the retractor blade configured to transfer a force applied to the retractor blade to the load cell via the coupler; and
a controller in communication with the load cell, the controller configured to modify the load cell signal.

12. The instrument of claim 11, further comprising:
a user interface in communication with the controller and configured to produce a display based on the load cell signal from the controller.

13. The instrument of claim 11, wherein the controller is configured to produce an alert based on the load cell signal when a threshold force is exceeded for a threshold time.

14. The instrument of claim 13, wherein the threshold force and the threshold time are indicative of a likelihood of creating post-operative pain.

15. The instrument of claim 13, wherein the controller is configured to transmit a control signal to a surgical arm connected to the end effector coupler to reduce the force applied to the retractor when the threshold force is exceeded for the threshold time.

16. The instrument of claim 13, wherein the controller is configured to transmit a control signal to a surgical arm connected to the end effector coupler to reduce the force applied to the retractor when a second threshold force is exceeded for a second threshold time.

17. The instrument of claim 13, wherein the controller is configured to control a color or intensity, based on the load signal, of a light emitted by a light connected to the retractor blade.

18. An instrument receivable in an end effector coupler of a surgical arm, the instrument comprising:
a stem including a proximal portion couplable to an end effector coupler, and an opposite distal portion;
a coupler including a mating feature; and
a retractor blade couplable to the coupler via the mating feature, the retractor blade, together with the stem and the coupler, movable with the end effector coupler.

19. The instrument of claim 18, wherein:
the mating feature of the coupler comprises a slot; and
a proximal portion of the retractor blade comprises a raised boss insertable into the slot of the mating feature and translatable therein to move the retractor blade between a locked position and an unlocked position with respect to the coupler.

20. The instrument of claim 18, further comprising:
a set screw positionable through the coupler into the slot, the set screw adjustable to engage the raised boss to limit translation of the boss within the slot.
